(19) United States
(12) Patent Application Publication
(10) Pub. No.: US 2008/0306356 A1
(43) Pub. Date: Dec. 11, 2008

(54) Vascular status monitoring system

(76) Inventor: Kenneth Darryl Kemp, Philadelphia, PA (US)

Correspondence Address: JOHN F. LETCHFORD, ARCHER & GREINER, P.C., ONE CENENNIAL SQUARE, HADDONFIELD, NJ, 08033, US

(21) Appl. No.: 12/156,934

(22) Filed: Jun. 5, 2008

Related U.S. Application Data
(60) Provisional application No. 60/942,153, filed on Jun. 5, 2007.

(51) Int. Cl. A61B 5/02

(52) U.S. Cl. 600/301

(57) ABSTRACT

A system including an adjustable strap having at least one sensor for monitoring a physical condition of a patient. The strap bears measurement indicia so that a clinician can establish baseline perimetral measurements of a patient's appendage and later account for changes in swelling that may be attributable to inflammation or edema. The system further includes a user-interactive unit for receiving, storing and processing patient information from the at least one sensor. In addition, the system desirably includes standardized forms for documenting a patient's physical health as a function of his/her sensed physical condition.

### CROSS REFERENCE TO RELATED APPLICATIONS

This application claims the benefit of U.S. Provisional Application Nos. 60/892,218, filed Feb. 28, 2007 and 60/942,153, filed Jun. 5, 2007.

### FIELD OF THE INVENTION

The present invention relates in general to medical apparatus and in particular to non-invasive systems and methods for monitoring a patient's vascular performance.

### BACKGROUND OF THE INVENTION

Non-invasive vascular monitoring devices are well known in the art. They typically monitor, without limitation, a patient's temperature, pulse, oxygen saturation (SpO₂%) and waveform and are commonly embodied as spring-biased clamp monitors that are releasably attachable to a person's fingers or toes. While useful for digital perfusion monitoring, such apparatus are of little use in providing circulation status at more proximal sites (midfoot, ankle, calf, thigh, hand, wrist, forearm, upper arm, etc.) that may be of particular interest to vascular, orthopedic, podiatric or other physicians or surgeons having need for timely and accurate proximal vascular status information. Digital monitors or probes are also of little value in situations where there may be gangrenous changes and the need to determine more proximal perfusion status is warranted.

### SUMMARY OF THE INVENTION

The present invention comprises a system including an adjustable strap having at least one sensor for monitoring a physical condition of a patient. The strap wraps around a patient's appendage and bears measurement indicia so that a clinician can establish baseline perimetral measurements of a patient's appendage and later account for changes in swelling that may be attributable to inflammation or edema. (As used herein, the term “appendage” shall be construed to be something other than a digit such as a finger or toe.) The system further preferably includes a portable, user-interactive unit for receiving, storing and processing patient information from the at least one sensor. In addition, the system desirably includes use of standardized forms for documenting a patient's long-term physical health as a function of his/her sensed physical condition.

Other details, objects and advantages of the present invention will become apparent as the following description of the presently preferred embodiments and presently preferred methods of practicing the invention proceeds.

### BRIEF DESCRIPTION OF THE DRAWINGS

The invention will become more readily apparent from the following description of preferred embodiments thereof shown, by way of example only, in the accompanying drawings wherein:

FIG. 1 is an example of a form according to the present invention for documenting a patient's physical health as a function of his/her sensed physical condition;

FIG. 2 is a perspective view of a portable, user-interactive patient information receiving, storing and processing unit according to the present invention; and

FIG. 3 is a perspective view of an adjustable strap according to the present invention having at least one sensor for monitoring a physical condition of a patient.

### DETAILED DESCRIPTION OF THE INVENTION

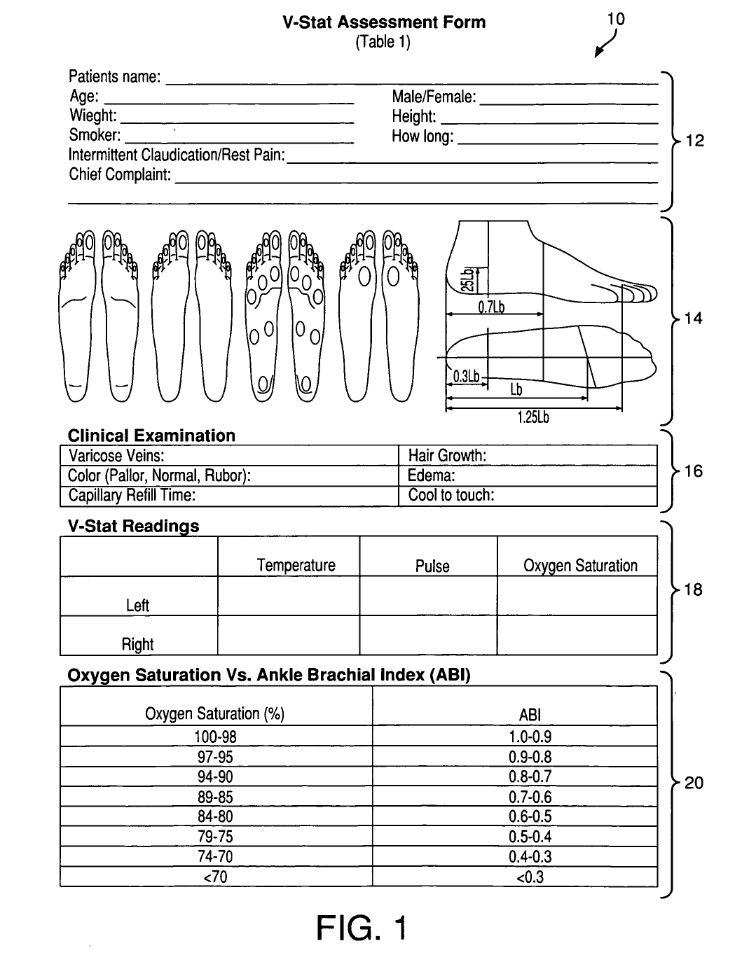

Referring to the drawings wherein like or similar references indicate like or similar elements throughout the several views, there is shown in FIG. 1 a vascular status (“V-Stat”) assessment form in accordance with the present invention. The form, identified generally by reference numeral 10, preferably includes a first section 12 for recording bibliographic information such as patient name, age, sex, weight, height, smoker status, chief complaint and the like. The V-Stat assessment form 10 shown in FIG. 1 is particularly directed to foot problems such as those that might be of interest to a podiatrist. However, form 10 may just as easily be tailored for hand specialists, orthopedists, vascular specialists or other health care professionals having need for information pertaining to other body appendages.

Form 10 preferably includes a second section 14 containing one or more images of the body part under scrutiny upon which medical personnel may take notes or draw images relating to a patient's specific physical problem. Form 10 further desirably includes a third section 16 for inputting other observable quantitative and/or qualitative clinical information such as the presence of varicose veins, color (pallor, normal, rubor), capillary refill time, hair growth, edema, “cool to touch”, etc.

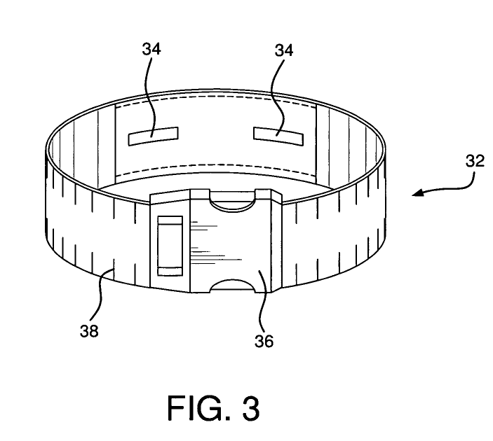

Form 10 additionally desirably includes a section 18 for inputting sensor-gathered physiological data such as, for example, temperature, pulse, oxygen saturation and the like. A presently preferred means for gathering such information is described in greater detail in connection with the description of FIG. 3, below. Lastly, form 10 preferably includes an empirical index or table section 20 (in the illustrated, although non-limitative case of FIG. 1, an Oxygen Saturation vs. Ankle Brachial Index) to assist the clinician in readily assessing the vascular status of a particular body part. It will be readily appreciated that such a table or index may be established for any desired appendage.

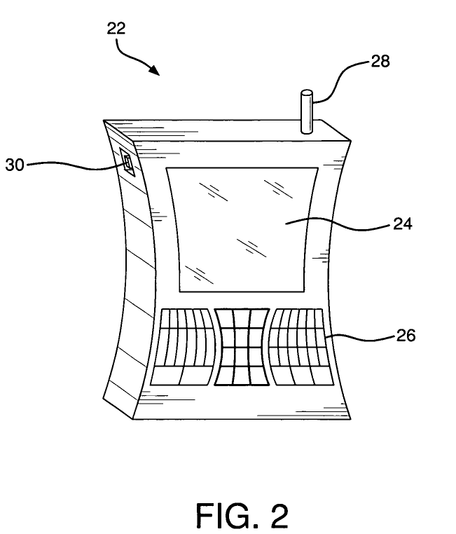

FIG. 2 depicts a perspective view of a user-interactive patient information receiving, storing and processing unit according to the present invention. The unit, identified generally by reference numeral 22, may be any suitable workstation, laptop, PC or other computer. Preferably, however, unit 22 is constructed and functions similarly to a lightweight, portable digital assistant (PDA) type device. Unit 22 is preferably both AC and DC powerable and includes a monitor or display 24 and an input/output device (I/O device) 26 such as a keyboard or the like having conventional alphanumeric keys and/or dedicated function keys by which a user such as a physician, clinician, nurse or patient may input data and respond to information observed on display 24. Unit 22 may include an external antenna 28 or an internal antenna. In this regard, unit 22 is preferably fully wirelessly capable and receives data continuously or in batch form from at least one physiological sensor borne by an adjustable patient-borne strap described in detail in respect to FIG. 3. Additionally, unit 22 is preferably equipped with a USB or similar presently known or hereinafter developed information transmission port 30 whereby a USB memory key or similar device may be used, if desired, to retrieve and save physiological information stored on the unit. In turn, information so saved on the USB memory key or similar device may then be downloaded and saved to a computer in the manner well known in the art.

FIG. 3 is a perspective view of an adjustable strap according to the present invention having at least one sensor for monitoring a physical condition of a patient. The strap, identified generally by reference numeral 32, may be fabricated from any suitable flexible medical-grade plastic, foam and/or fabric materials (or comparable materials) that are durable in construction and capable of easy cleaning/disinfection. Strap 32 preferably includes one or more presently known or hereinafter developed physiological sensors 34 such as light emitting diodes (LEDs), electrodes or the like that are useful for use in detecting oxygen saturation, pulse, temperature and/or other physiological characteristics of a patient. Strap 32 includes means 36 for releasably and adjustably securing the strap about the desired body part. Such means may include, without limitation, any suitable adjustment means such as belt clasps, hook and loop type fasteners, snaps, buttons and so on. In addition, strap 32 includes measurement indicia 38, preferably in both English and metric units, for enabling the patient and/or his or her medical service provider to obtain baseline and subsequent treatment readings of the appendage under scrutiny so that a clinician can establish baseline perimetral measurements of a patient's appendage and later account for changes in swelling that may be attributable to inflammation or edema.

Although not illustrated and described in detail because such information transmission technology is well known to those skilled in the art, strap 32 may be equipped with a miniature transmitter and power supply sufficient to wirelessly transfer data recorded by the sensor(s) 34 to an Internet server whereby the information recorded by the sensor(s) 34 may be received by remote unit 22.

It will be understood that the form 10 shown in FIG. 1 is merely representative of but one example of how such a form might appear in a paper layout and format. It will likewise be understood that form 10 may assume any desired format so long as it contains bibliographic and clinical information sufficient to document a patient's identity and physical condition consistent with input provided by a health care worker and/or sensor(s) 34. Furthermore, regardless of whatever format it may assume and content it may contain, form 10 may be electronically stored in unit 22 and any or all patient information may be physically input into the electronic form by a person such as a health care professional (or a patient) via I/O device 26. Alternatively, some patient vascular information may be automatically input into the electronic form responsive to data readings recorded by sensor(s) 34 borne by strap 32 and transmitted to unit 22. Regardless of how the form 10 may appear on display 24 of unit 22, and regardless of how information may be introduced into the form, it will be understood that the form and the information contained therein may be downloaded, stored and/or printed by means well known in the art.

Although the invention has been described in detail for the purpose of illustration, it is to be understood that such detail is solely for that purpose and that variations can be made therein by those skilled in the art without departing from the spirit and scope of the invention as claimed herein.

What is claimed is:

1. A vascular status monitoring system comprising:
an adjustable strap adapted for wrapping about an appendage of a patient, said strap carrying at least one physiological sensor for monitoring at least one physiological characteristic of the patient;
a computer in communication with said at least one physiological sensor; and
a form for recording physiological data monitored by said at least one physiological sensor.

2. The system of claim 1 wherein said computer comprises a portable computer.

3. The system of claim 1 wherein said form is a paper form.

4. The system of claim 1 wherein said form is an electronic form stored on said computer.
